(19) United States
(12) Patent Application Publication
(10) Pub. No.: US 2019/0083847 A1
(43) Pub. Date: Mar. 21, 2019

(54) CONTROL METHOD AND CONTROL DEVICE OF EXERCISE DEVICE, AND EXERCISE DEVICE

(71) Applicant: BOE Technology Group Co., Ltd., Beijing (CN)

(72) Inventor: Xiao Chu, Beijing (CN)

(21) Appl. No.: 16/033,355

(22) Filed: Jul. 12, 2018

(30) Foreign Application Priority Data
Sep. 20, 2017 (CN) 201710855238.6

(51) Int. Cl. A63B 24/00; A61B 5/11; A63B 22/02

(52) U.S. Cl. CPC A63B 24/0087; A63B 24/0062; A61B 5/1128; A63B 22/02; A63B 2024/0093; A63B 2220/806; A63B 2220/62; A61B 2560/0475; A63B 2220/30

(57) ABSTRACT

A control method of an exercise device includes: detecting body movements of an exerciser; acquiring exercise parameters of the exerciser in outdoor exercises according to a mapping relationship between the body movements of the exerciser and the exercise parameters of the exerciser in the outdoor exercises; and driving the exercise device to operate according to the exercise parameters.

### CROSS-REFERENCE TO RELATED APPLICATION

This application claims the benefit of Chinese Patent Application No. 201710855238.6 filed on Sep. 20, 2017 in the State Intellectual Property Office of China, the whole disclosure of which is incorporated herein by reference.

### TECHNICAL FIELD

The present disclosure relates to exercise device technologies, and more particularly, to a control method of an exercise device, a control device of an exercise device and an exercise device.

### BACKGROUND

With the improvement of quality of life, exercise and fitness become more and more popular, and increasing numbers of people can be found exercising outdoors (e.g., walking, running, biking) and indoors (e.g., on treadmills, stationary bikes, elliptical machines).

There is a contradiction for exerciser. On the one hand, exercises on exercise devices such as treadmills and the like are not as healthy and comfortable as outdoor exercises. On the other hand, the exerciser has to exercise indoors in bad weather (such as rain, snow, wind, fog, etc.). Unfortunately, exercises on the exercise devices such as treadmills may disrupt an exercise rhythm of the exerciser, resulting in that the exerciser is unable to keep his/her outdoor exercise habit.

### SUMMARY

In an aspect, it is provided a control method of an exercise device, including:

detecting body movements of an exerciser,

acquiring exercise parameters of the exerciser in outdoor exercises according to a mapping relationship between the body movements of the exerciser and the exercise parameters of the exerciser in the outdoor exercises; and

driving the exercise device to operate according to the exercise parameters.

Optionally, the exercise parameters include at least one selected from exercise scenes, exercise speeds and exercise durations.

Optionally, the mapping relationship includes a first mapping relationship between the body movements of the exerciser and the exercise scenes of the exerciser in the outdoor exercises, and

wherein the acquiring exercise parameters of the exerciser in outdoor exercises according to a mapping relationship between the body movements of the exerciser and the exercise parameters of the exerciser in the outdoor exercises includes:

detecting the body movements of the exerciser in sequence, during a pre-recorded video in which the exerciser is exercising in the exercise scenes of the outdoor exercises is played or a pre-captured image in which the exerciser is exercising in the exercise scenes of the outdoor exercises is played; and

acquiring the exercise scenes corresponding to detected body movements of the exerciser according to the first mapping relationship between the body movements of the exerciser and the exercise scenes of the exerciser in the outdoor exercises, in response to detecting the body movements of the exerciser.

Optionally, the driving the exercise device to operate according to the exercise parameters includes:

driving the exercise device to operate according to the exercise scenes.

Optionally, the exercise parameters include multiple sets of exercise parameters, each set of exercise parameters including an exercise scene, an exercise speed corresponding to the exercise scene and an exercise duration corresponding to the exercise scenes, and

wherein the driving the exercise device to operate according to the exercise parameters includes:

according to an acquired exercise scene, determining the exercise speed corresponding to the acquired exercise scene and the exercise duration corresponding to the acquired exercise scene; and

driving the exercise device to operate in a set of exercise parameters, the set of exercise parameters including the acquired exercise scene, the exercise speed corresponding to the acquired exercise scene and the exercise duration corresponding to the acquired exercise scene.

Optionally, the mapping relationship includes a second mapping relationship between the body movements of the exerciser and the exercise speeds of the exerciser in the outdoor exercises, and

wherein the acquiring exercise parameters of the exerciser in outdoor exercises according to a mapping relationship between the body movements of the exerciser and the exercise parameters of the exerciser in the outdoor exercises includes:

detecting the body movements of the exerciser in sequence, during a pre-recorded video in which the exerciser is exercising in the exercise scenes of the outdoor exercises is played or a pre-captured image in which the exerciser is exercising in the exercise scenes of the outdoor exercises is played; and

acquiring exercise speeds corresponding to detected body movements of the exerciser according to the second mapping relationship between the body movements of the exerciser and the exercise speeds of the exerciser in the outdoor exercises, in response to detecting the body movements of the exerciser.

Optionally, the driving the exercise device to operate according to the exercise parameters includes: driving the exercise device to operate according to the exercise speeds.

Optionally, after the acquiring the exercise scenes corresponding to detected body movements of the exerciser according to the first mapping relationship between the body movements of the exerciser and the exercise scenes of the exerciser in the outdoor exercises in response to detecting the body movements of the exerciser, the method further includes:

calculating a time interval between a moment when a body movement of the exerciser is currently detected and another moment when a body movement of the exerciser was once detected most recently;

determining the time interval as an exercise duration of one of the exercise scenes, the one of the exercise scenes corresponding to the body movement of the exerciser which was once detected most recently; and

recording a third mapping relationship between the exercise scenes and the exercise durations.

Optionally, the driving the exercise device to operate according to the exercise scenes includes:

driving the exercise device to operate in a first operating state according to an acquired first exercise scene;

determining a first exercise duration corresponding to the first exercise scene according to the third mapping relationship; and

driving the exercise device to operate in a second operating state different from the first operating state, after the exercise device operates in the first operating state for the first exercise duration.

In another aspect, it is provided an exercise device, including:

a movement sensing device configured to detect body movements of an exerciser;

a control device including:
a memory;
a processor;
one or more modules stored in the memory and configured to be executed by the processor, the one or more modules including instructions:
   to acquire exercise parameters of the exerciser in outdoor exercises according to a mapping relationship between the body movements of the exerciser and the exercise parameters of the exerciser in the outdoor exercises; and
   to drive the exercise device to operate according to the exercise parameters.

Optionally, the exercise parameters include at least one selected from exercise scenes, exercise speeds and exercise durations.

Optionally, the mapping relationship includes a first mapping relationship between the body movements of the exerciser and the exercise scenes of the exerciser in the outdoor exercises, and

wherein the instruction to acquire exercise parameters of the exerciser in outdoor exercises according to a mapping relationship between the body movements of the exerciser and the exercise parameters of the exerciser in the outdoor exercises includes:

detecting the body movements of the exerciser in sequence, during a pre-recorded video in which the exerciser is exercising in the exercise scenes of the outdoor exercises is played or a pre-captured image in which the exerciser is exercising in the exercise scenes of the outdoor exercises is played; and

acquiring the exercise scenes corresponding to detected body movements of the exerciser according to the first mapping relationship between the body movements of the exerciser and the exercise scenes of the exerciser in the outdoor exercises, in response to detecting the body movements of the exerciser.

Optionally, the instruction to drive the exercise device to operate according to the exercise parameters includes:

driving the exercise device to operate according to the exercise scenes.

Optionally, the exercise parameters include multiple sets of exercise parameters, each set of exercise parameters including an exercise scene, an exercise speed corresponding to the exercise scene and an exercise duration corresponding to the exercise scene, and

wherein the instruction to drive the exercise device to operate according to the exercise parameters includes:

according to an acquired exercise scene, determining the exercise speed corresponding to the acquired exercise scene and the exercise duration corresponding to the acquired exercise scene; and

driving the exercise device to operate in a set of exercise parameters, the set of exercise parameters including the acquired exercise scene, the exercise speed corresponding to the acquired exercise scene and the exercise duration corresponding to the acquired exercise scene.

Optionally, the mapping relationship includes a second mapping relationship between the body movements of the exerciser and the exercise speeds of the exerciser in the outdoor exercises, and

wherein the instruction to acquire exercise parameters of the exerciser in outdoor exercises according to a mapping relationship between the body movements of the exerciser and the exercise parameters of the exerciser in the outdoor exercises includes:

detecting the body movements of the exerciser in sequence, during a pre-recorded video in which the exerciser is exercising in the exercise scenes of the outdoor exercises is played or a pre-captured image in which the exerciser is exercising in the exercise scenes of the outdoor exercises is played; and

acquiring exercise speeds corresponding to detected body movements of the exerciser according to the second mapping relationship between the body movements of the exerciser and the exercise speeds of the exerciser in the outdoor exercises, in response to detecting the body movements of the exerciser.

Optionally, the instruction to drive the exercise device to operate according to the exercise parameters includes:

driving the exercise device to operate according to the exercise speeds.

Optionally, the one or more modules further include instructions:

to calculate a time interval between a moment when a body movement of the exerciser is currently detected and another moment when a body movement of the exerciser was once detected most recently;

to determine the time interval as an exercise duration of one of the exercise scenes, the one of the exercise scenes corresponding to the body movement of the exerciser which was once detected most recently; and

to record a third mapping relationship between the exercise scenes and the exercise durations.

Optionally, the instruction to drive the exercise device to operate according to the exercise scenes includes:

driving the exercise device to operate in a first operating state according to an acquired first exercise scene;

determining a first exercise duration corresponding to the first exercise scene according to the third mapping relationship; and

driving the exercise device to operate in a second operating state different from the first operating state, after the exercise device operates in the first operating state for the first exercise duration.

In a further aspect, it is provided a control device of an exercise device, including:

a scene recording device configured to record exercise parameters of an exerciser in outdoor exercises;

a movement sensing device configured to detect body movements of the exerciser; and

a control device including:
a memory;
a processor;
one or more modules stored in the memory and configured to be executed by the processor, the one or more modules including instructions:
   to acquire exercise parameters of the exerciser in outdoor exercises according to a mapping relationship between the body movements of the exerciser and the exercise parameters of the exerciser in the outdoor exercises; and
   to drive the exercise device to operate according to the exercise parameters.

In another further aspect, it is provided a computer readable storage medium on which computer program instructions are stored, wherein the computer program instructions are executed to implement steps of the control method of the exercise device as described above.

### BRIEF DESCRIPTION OF THE DRAWINGS

Accompanying drawings are to be used to provide a further understanding of the present disclosure and to form a part of the specification, and together with the embodiments of the present application, serve to explain technical schemes of the present disclosure and are not to be construed as limiting the technical schemes of the present disclosure.

FIG. 1 is a flow chart of a control method of an exercise device according to an exemplary embodiment of the present disclosure;

FIG. 2 is a flow chart of a control method of an exercise device according to an exemplary embodiment of the present disclosure;

FIG. 3 is a block diagram of an exercise device according to an exemplary embodiment of the present disclosure;

FIG. 4 is a block diagram of an exercise device according to an exemplary embodiment of the present disclosure;

FIG. 5 is a block diagram of a computer readable storage medium according to an exemplary embodiment of the present disclosure; and

FIG. 6 is a block diagram of a control device of an exercise device according to an exemplary embodiment of the present disclosure.

### DETAILED DESCRIPTION OF THE EMBODIMENTS

Embodiments of the present disclosure will be described in detail hereinafter with reference to the accompanying drawings. It should be noted that the embodiments of the present disclosure and features in the embodiments may be combined with each other without conflict.

Steps shown in the flowcharts of the drawings may be performed in a computer system, such as a set of computer executable instructions. Also, while a logical order is shown in the flowcharts, in some cases the steps shown or described may be performed in a different order from the logical order.

It should be noted that the following detailed descriptions of the present disclosure are mainly described by taking running exercise as an example, however, those skilled in the art will appreciate that the embodiments of the present disclosure are not limited to running exercise, and may also be applied to other various types of exercises such as walking, biking, skiing, climbing and the like. Accordingly, the exercise devices mentioned herein may be treadmills or other types of exercise devices, such as exercise bikes, elliptical machines, skiing exercise devices, rowing machines, steppers, hikers, climbers and the like.

Unless otherwise noted, “exercise scene” herein generally refers to scenes in which the exerciser is exercising, including but not limited to, flat road, uphill, downhill, left turn, right turn, uphill slope, down slope, etc.

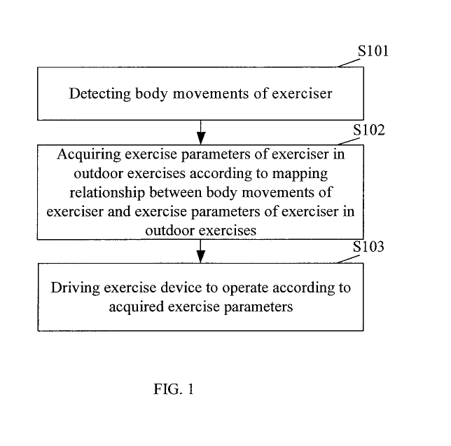

Referring to FIG. 1, a control method of an exercise device according to an exemplary embodiment of the present disclosure may include the following steps.

In step S101, body movements of the exerciser may be detected.

For example, the body movements of the exerciser may be predefined, including but not limited to, head raising, head lowering, head shaking, leftward head rotating, rightward head rotating, arm swinging, double head raising, double head lowering, double head shaking, double leftward head rotating, double rightward head rotating, double arm swinging and the like. In the embodiments of the present disclosure, by means of double head raising, double head lowering, double head shaking, double leftward head rotating, double rightward head rotating, double arm swinging and the like, the body movements may be detected without interference by operations such as perspiration wiping and the like. The body movements of the exerciser may include not only types of the body movements, but also extents of the body movements, that is, the body movements of the exerciser may include a combination of the movement types and the movement extents, for example, the body movements of the exerciser may include a combination of the double head raising and a head raising extent, a combination of the double head lowering and a head lowering extent, a combination of the double arm swinging and an arm swinging extent and the like.

For example, the body movements of the exerciser may be detected by the movement sensing device. For example, the movement sensing device may include a hardware such as camera or the like, and be configured to detect the body movements of the exerciser by means of motion capturing techniques. The motion capturing techniques in the related art may be employed to detect the body movements of the exerciser, and will not be described in detail herein.

In step S102, exercise parameters of the exerciser in outdoor exercises may be acquired according to a mapping relationship between the body movements of the exerciser and the exercise parameters of the exerciser in the outdoor exercises.

For example, the exercise parameters may include at least one selected from exercise scene, exercise speed and exercise duration.

In one example, the mapping relationship between the body movements of the exerciser and the exercise parameters of the exerciser in the outdoor exercises may be shown as follows.

TABLE 1
Mapping relationship between body movements and exercise parameters

| body movements | exercise parameters |
|---|---|
| double head shaking | flat road |
| double head raising | uphill |
| double head lowering | downhill |
| double leftward head rotating | left turn |
| double rightward head rotating | right turn |
| combination of double head raising and | uphill slope |
| head raising extent | |
| combination of double head lowering and | downhill slope |
| head lowering extent | |
| combination of double arm swinging and | exercise speed |
| arm swinging extent | |

In one example, the mapping relationship between the body movements of the exerciser and the exercise parameters of the exerciser in the outdoor exercises may include a first mapping relationship between the body movements of the exerciser and the exercise scenes of the exerciser in the outdoor exercises. For example, referring to Table 1, the double head shaking, the double heading raising, the double head lowering, the double leftward head rotating and the double rightward head rotating of the exerciser may correspond to exercise scenes such as the flat road, the uphill, the downhill, the left turn and the right turn, respectively. Referring to Table 1, the body movements of the exerciser may include a combination of the body movement type and the body movement extent. For example, the body movements of the exerciser may include the combination of the double head raising and the head raising extent, and the combination of the double head raising and the head raising extent may correspond to the uphill slope. Similarly, the combination of the double head lowering and the head lowering extent may correspond to the downhill slope.

In one example, the exercise parameters may be presented in combination, for example, the exercise parameters may include multiple sets of exercise parameters, each set of exercise parameters including an exercise scene, an exercise speed corresponding to the exercise scene and an exercise duration corresponding to the exercise scene. The multiple sets of exercise parameters may be predefined and stored in the exercise device. In the example, the exercise scene corresponds to other parameters (e.g., exercise speed, exercise duration, etc.), so that the exercise scene may correspond to an index term. The exercise scene may be acquired according to the first mapping relationship between the body movements of the exerciser and the exercise scenes of the exerciser in the outdoor exercises, and then the other corresponding parameters are acquired according to the exercise scene.

In one example, the mapping relationship between the body movements of the exerciser and the exercise parameters of the exerciser in the outdoor exercises may include a second mapping relationship between the body movements of the exerciser and the exercise speed of the exerciser in the outdoor exercises. For example, referring to Table 1, the body movements of the exerciser may include the combination of the double arm swinging and the arm swinging extent, and the combination of the double arm swinging and the arm swinging extent may correspond to the exercise speed such as running speed.

According to an exemplary embodiment of the present disclosure, the exercise scenes of the exerciser in the outdoor exercises may be acquired by the following means.

First, a pre-recorded video in which the exerciser is exercising in the exercise scene of the outdoor exercise is played or a pre-captured image in which the exerciser is exercising in the exercise scene of the outdoor exercise is played. During the exerciser watches the video or the image, each time the exercise scene is found to be changed, the exerciser may make the body movement which corresponds to a changed exercise scene, and the body movements made by the exerciser are detected in sequence. Then, each time the body movement made by the exerciser is detected, the exercise scene corresponding to the detected body movement is acquired according to the predefined first mapping relationship between the body movements and the exercise scenes.

Alternatively, the video may be a video that is recorded in real time by the exerciser in the exercise scenes in which the exerciser is used to exercise. The image may be an image that is captured in real time by the exerciser in the exercise scenes in which the exerciser is used to exercise. When the image is captured, periodic or non-periodic timing capturing may be employed.

According to some embodiments, the video or image may be sent to the exercise device (e.g., treadmill) from a scene recording device in a wired or wireless manner (e.g., Bluetooth, WiFi, etc.) or may be input into the exercise device by a user, after the scene recording device captures the video or image.

For example, the scene recording device may be a wearable device such as intelligent glasses or the like, or may be a mobile terminal such as a smart phone or the like. The scene recording device may also be other devices which may record the video or capture the image, and the embodiments of the present disclosure are not intended to limit the scene recording device. Advantageously, by using the wearable devices such as intelligent glasses, the exerciser may record video or capture image without affecting normal exercises.

According to an exemplary embodiment of the present disclosure, the exercise speed of the exerciser in the outdoor exercises may be acquired in the following manner.

First, a pre-recorded video in which the exerciser is exercising in the exercise scene of the outdoor exercise is played or a pre-captured image in which the exerciser is exercising in the exercise scene of the outdoor exercise is played. During the exerciser watches the video or the image, the body movements made by the exerciser are detected in sequence.

Then, each time the body movement made by the exerciser is detected, the exercise speed corresponding to the detected body movement is acquired according to the predefined second mapping relationship between the body movements and the exercise speeds. For example, different arm swinging extents are used to represent different exercise speeds, so that the exercise device may acquire different exercise speeds according to the detected arm swinging extents.

Alternatively, the exercise speeds may be detected when the exerciser is in the outdoor exercises.

In step S103, the exercise device is driven to operate according to the acquired exercise parameters.

For example, the exercise device may be driven to operate according to the acquired exercise scenes. In other words, an operating state of the exercise device may be changed in sequence according to all the acquired exercise scenes. For example, when the acquired exercise scene is uphill, the treadmill may lift a front of its movable member. When the acquired exercise scene is downhill, the treadmill may lift a rear of the movable member. When the acquired exercise scene is left turn, the treadmill may tilt the movable member leftward. When the acquired exercise scene is right turn, the treadmill may tilt the movable member rightward.

For example, the exercise parameters may be presented in combination, for example, the exercise parameters may include multiple sets of exercise parameters, each set of exercise parameters including an exercise scene, an exercise speed corresponding to the exercise scene and an exercise duration corresponding to the exercise scene. The multiple sets of exercise parameters may be predefined and stored in the exercise device. In this case, the step S103 may specifically include: according to an acquired exercise scene, determining the exercise speed corresponding to the acquired exercise scene and the exercise duration corresponding to the acquired exercise scene; and driving the exercise device to operate in the exercise parameters which includes the acquired exercise scene, the exercise speed corresponding to the acquired exercise scene and the exercise duration corresponding to the acquired exercise scene.

For example, the exercise device may be operated in a predefined speed after its operating state is changed according to the exercise scenes. For example, the speed of the exercise device itself may be changed according to a speed of the exerciser on the exercise device, for example, by rotating the movable member under the drive of a friction force between the exerciser and the movable member.

In order to copy an outdoor exercise habit into indoor exercises better, the movable member of the exercise device may be driven to rotate in accordance with the exercise speeds of the exerciser upon recording the video or capturing the image. Other ways may also be used to set the exercise speeds of the exercise device.

For example, the exercise speeds may be acquired according to the second mapping relationship between the body movements of the exerciser and the exercise speeds of the exerciser in the outdoor exercises. In this case, the step S103 may specifically include: driving the exercise device to operate according to the acquired exercise speed.

For example, when the operating states of the exercise device are changed according to the exercise scenes, a time interval between two adjacent operating states of the exercise device may be preset.

In order to copy the outdoor exercise habit to indoor exercises better, for example, the time interval between two adjacent operating states may be set according to an actual time interval between two adjacent exercise scenes upon recording the video or capturing the image.

For example, each time the body movement made by the exerciser is detected, the exercise scene corresponding to the detected body movement is acquired according to the first mapping relationship between the body movements of the exerciser and the exercise scenes of the exerciser in the outdoor exercises. Thereafter, the method may further include: calculating a time interval between a moment when a body movement of the exerciser is currently detected and another moment when a body movement of the exerciser was previously detected (for example, when a body movement of the exerciser was once detected most recently); determining that the time interval as the exercise duration of the exercise scene which corresponds to the body movement of the exerciser which was previously detected (for example, when a body movement of the exerciser was once detected most recently); and recording a third mapping relationship between the exercise scenes and the exercise durations.

Accordingly, the step S103 may specifically include:

driving the exercise device to operate in a first operating state according to an acquired first exercise scene;

determining a first exercise duration corresponding to the first exercise scene according to the third mapping relationship; and

driving the exercise device to operate in a second operating state different from the first operating state, after the exercise device operates in the first operating state for the first exercise duration.

It should be noted that the video may be recorded by the scene recording device when the exerciser is in the outdoor exercises and played by the scene recording device, and the image may be captured by the scene recording device when the exerciser is in the outdoor exercises and played by the scene recording device.

Alternatively, the video may be recorded by the scene recording device when the exerciser is in the outdoor exercises and played by the exercise device, and the image may be captured by the scene recording device when the exerciser is in the outdoor exercises and played by the exercise device.

By means of the embodiments of the present disclosure, all the exercise scenes, exercise speeds and/or exercise durations of the exerciser in the outdoor exercises may be input into the exercise device, then the exercise device may change its operating states according to all the exercise scenes, exercise speeds and/or exercise durations when the exerciser needs to perform indoor exercises. In this way, the habits of the exerciser in the outdoor exercises may be copied into the indoor exercises, thereby coordinating the exercise habits of the exerciser in the outdoor exercises with the exercise habits of the exerciser in the indoor exercises, and improving the user experience.

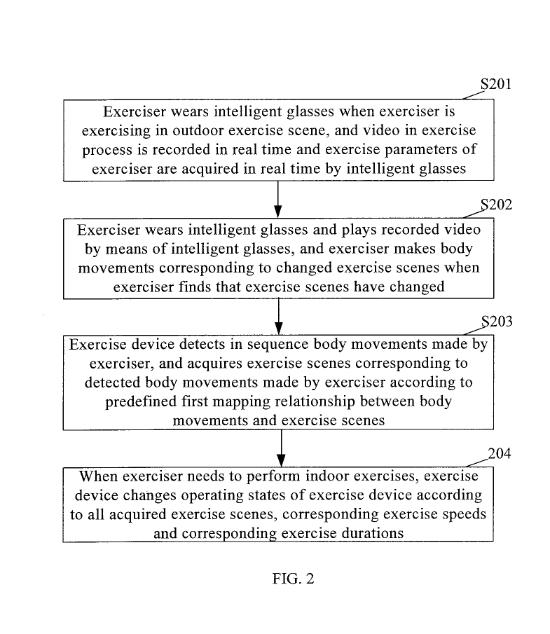

Referring to FIG. 2, a control method of an exercise device according to an exemplary embodiment of the present disclosure is shown. Various steps of the control method are described in detail below by taking the running exercise as an example.

In step S201, the exerciser wears intelligent glasses when the exerciser is exercising in the outdoor exercise scene, and the video in the exercise process is recorded in real time and the exercise parameters of the exerciser are acquired in real time by the intelligent glasses, the exercise parameters including but not limited to exercise scenes, exercise speeds and exercise durations.

For example, when the exerciser may run in a certain park at 17 o'clock, the exercise scene in which the exerciser is running from 17 o'clock to 17:10 is flat road, the exercise scene in which the exerciser is running from 17:10 to 17:12 is downhill, the exercise scene in which the exerciser is running from 17:12 to 17:15 is flat road, the exercise scene in which the exerciser is running from 17:15 to 17:16 is left turn, the exercise scene in which the exerciser is running from 17:15 to 17:26 is flat road, the exercise scene in which the exerciser is running from 17:26 to 17:28 is uphill, the exercise scene in which the exerciser is running from 17:28 to 17:33 is flat road, the exercise scene in which the exerciser is running from 17:33 to 17:34 is right turn, and the exercise scene in which the exerciser is running from 17:34 to 17:44 is flat road. At this point, the running process ends and it lasts 44 minutes.

for example, the exercise speed in flat road is 3 kilometers per hour (km/h), the exercise speed in downhill is 4 km/h, the exercise speed in uphill is 2 km/h, and the exercise speed in left turn and the exercise speed in right turn are 1 km/h.

In step S202, the exerciser wears the intelligent glasses and plays the recorded video by means of the intelligent glasses, and the exerciser may make body movements corresponding to changed exercise scenes when the exerciser finds that the exercise scenes have changed.

For example, the exercise scenes which are presented in the video in sequence include flat road, downhill, flat road, left turn, flat road, uphill, flat road, right turn and flat road.

Then, the following body movements may be made in sequence according to the predefined first mapping relationship between the body movements and the exercise scenes: double head shaking, double head lowering, double head shaking, double leftward head rotating, double head shaking, double head raising, double head shaking, double rightward head rotating and double head shaking.

The exercise speeds include in sequence: 3 km/h, 4 km/h, 3 km/h, 1 km/h, 3 km/h, 2 km/h, 3 km/h, 1 km/h, 3 km/h.

The exercise durations include in sequence: 10 minutes, 2 minutes, 3 minutes, 1 minute, 10 minutes, 2 minutes, 5 minutes, 1 minute and 10 minutes.

In step S203, the exercise device detects in sequence the body movements made by the exerciser, and acquires the exercise scenes corresponding to the detected body movements made by the exerciser according to the predefined first mapping relationship between the body movements and the exercise scenes.

The exercise device may detect the following body movements in sequence: double head shaking, double head lowering, double head shaking, double leftward head rotating, double head shaking, double head raising, double head shaking, double rightward head rotating and double head shaking.

By searching the first mapping relationship, the following exercise scenes may be acquired: flat road, downhill, flat road, left turn, flat road, uphill, flat road, right turn and flat road.

Further, the acquired corresponding exercise durations are in sequence: 10 minutes, 2 minutes, 3 minutes, 1 minute, 10 minutes, 2 minutes, 5 minutes, 1 minute and 10 minutes. The corresponding exercise speeds are in sequence: 3 km/h, 4 km/h, 3 km/h, 1 km/h, 3 km/h, 2 km/h, 3 km/h, 1 km/h, 3 km/h.

In step S204, when the exerciser needs to perform the indoor exercises, the exercise device may change the operating states of the exercise device according to all the acquired exercise scenes, the corresponding exercise speeds and the corresponding exercise durations.

For example, in an initial period of the exercise, the movable member is kept parallel to the ground and the speed is 3 km/h. After 10 minutes, the rear of the movable member is tilted upward and the speed is 4 km/h. After 2 minutes, the movable member is changed to be parallel to the ground and the speed is 3 km/h. After 3 minutes, the movable member is tilted leftward and the speed is 1 km/h. After 1 minute, the movable member is changed to be parallel to the ground and the speed is 3 km/h. After 10 minutes, the front of the movable member is tilted upward and the speed is 2 km/h. After 2 minutes, the movable member is changed to be parallel to the ground and the speed is 3 km/h. After 5 minutes, the movable member is tilted rightward and the speed is 1 km/h. After 1 minute, the movable member is changed to be parallel to the ground and the speed is 3 km/h. After 10 minutes, the exercise device is stopped, and the indoor exercise is ended.

It should be noted that the above-described embodiment is merely one particular example of the control method of the exercise device according to an embodiment of the present disclosure, and is not intended to limit the scope of the present disclosure.

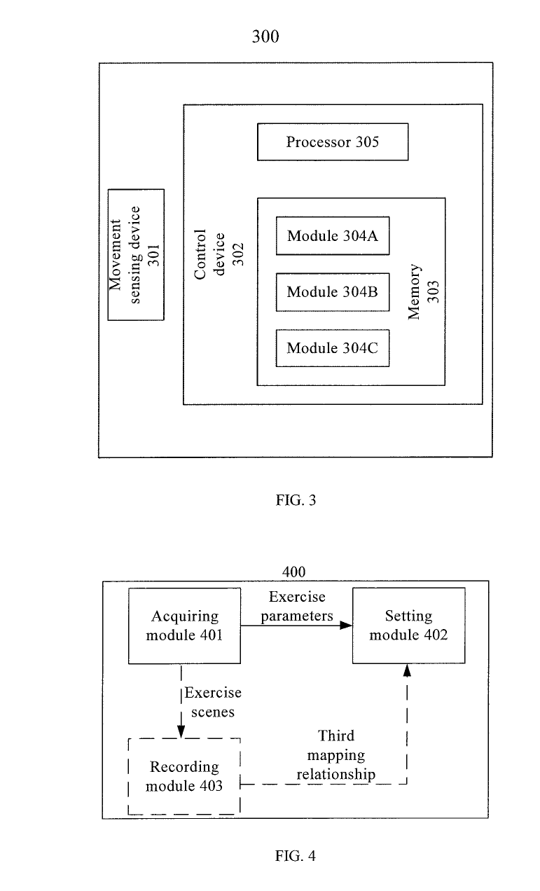

According to an exemplary embodiment of the present disclosure, an exercise device 300 is provided, as shown in FIG. 3, including: a movement sensing device 301 configured to detect the body movements of the exerciser; a control device 302 which may include a memory 303, a processor 305, and one or more modules 304A, 304B, 304C stored in the memory 303. The one or more modules 304A, 304B, 304C may be configured to be executed by the processor 305.

For example, the one or more modules 304A, 304B, 304C may include the following instructions: acquiring the exercise parameters according to the mapping relationship between the body movements of the exerciser and the exercise parameters of the exerciser in the outdoor exercises (e.g., the mapping relationship in Table 1); and driving the exercise device 300 to operate according to the acquired exercise parameters.

For example, the movement sensing device 301 may include a camera, or other sensors that may sense the body movements. The movement sensing device 301 may detect the body movements of the exerciser by means of motion capture techniques.

For example, the exercise parameters include, but are not limited to, exercise scenes, exercise speeds and exercise durations.

In one example, the one or more modules 304A, 304B, 304C may include the following instructions: during a pre-recorded video in which the exerciser is exercising in the exercise scene of the outdoor exercise is played or a pre-captured image in which the exerciser is exercising in the exercise scene of the outdoor exercise is played, detecting the body movements made by the exerciser in sequence; each time the body movement made by the exerciser is detected, acquiring the exercise scene corresponding to the detected body movement made by the exerciser according to the first mapping relationship between the body movements of the exerciser and the exercise scenes in the outdoor exercises.

In the example, the one or more modules 304A, 304B, 304C may also include the following instructions: driving the exercise device 300 to operate according to the acquired exercise scene.

In an example, the one or more modules 304A, 304B, 304C may include the following instructions: according to an acquired exercise scene, determining the exercise speed corresponding to the acquired exercise scene and the exercise duration corresponding to the acquired exercise scene; and driving the exercise device 300 to operate in the exercise parameters which includes the acquired exercise scene, the exercise speed corresponding to the acquired exercise scene and the exercise duration corresponding to the acquired exercise scene.

In an example, the one or more modules 304A, 304B, 304C may include the following instructions: during a pre-recorded video in which the exerciser is exercising in the exercise scene of the outdoor exercise is played or a pre-captured image in which the exerciser is exercising in the exercise scene of the outdoor exercise is played, detecting the body movements made by the exerciser in sequence; and each time the body movement made by the exerciser is detected, acquiring the exercise speed corresponding to the detected body movement according to the second mapping relationship between the body movements of the exerciser and the exercise speeds of the exerciser in the outdoor exercises.

In the example, the one or more modules 304A, 304B, 304C may include the following instructions: driving the exercise device to operate according to the acquired exercise speed. In an example, the one or more modules 304A, 304B, 304C may also include the following instructions: calculating a time interval between a moment when a body movement of the exerciser is currently detected and another moment when a body movement of the exerciser was previously detected (for example, when a body movement of the exerciser was once detected most recently); determining the time interval as the exercise duration of the exercise scene which corresponds to the body movement of the exerciser which was previously detected (for example, when a body movement of the exerciser was once detected most recently); and recording a third mapping relationship between the exercise scenes and the exercise durations.

In the example, the one or more modules 304A, 304B, 304C may include the following instructions: driving the exercise device to operate in a first operating state according to an acquired first exercise scene; determining a first exercise duration corresponding to the first exercise scene according to the third mapping relationship; and driving the exercise device to operate in a second operating state different from the first operating state, after the exercise device operates in the first operating state for the first exercise duration.

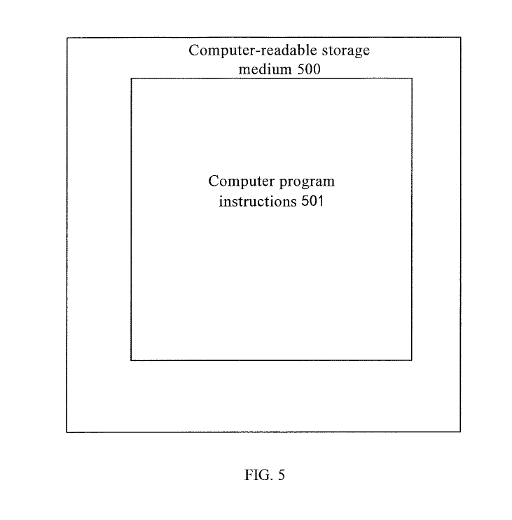

Referring to FIG. 4, a block diagram of an exercise device according to an exemplary embodiment of the present disclosure is schematically illustrated. The exercise device 400 may include: an acquiring module 401 or acquiring exercise parameters of an exerciser in outdoor exercises; and a setting module 402 for changing operating states of the exercise device according to the acquired exercise parameters when the exerciser needs to perform indoor exercises.

In an example, the acquiring module 401 may specifically acquire the exercise parameters of the exerciser in the outdoor exercises by the following manner: during a pre-recorded video in which the exerciser is exercising in the exercise scene of the outdoor exercise is played or a pre-captured image in which the exerciser is exercising in the exercise scene of the outdoor exercise is played, detecting the body movements made by the exerciser in sequence; each time the body movement made by the exerciser is detected, acquiring the exercise scene corresponding to the detected body movement made by the exerciser according to the predefined first mapping relationship between the body movements and the exercise scenes.

Accordingly, the setting module 402 may change the operating states of the exercise device according to the acquired exercise parameters by the following manner: changing the operating states of the exercise device in sequence according to all the acquired exercise scenes.

In an example, the exercise device 400 may also include a recording module 403 for recording a third mapping relationship between the exercise scenes and the exercise durations.

Referring to FIG. 5, there is also provided a computer-readable storage medium 500 on which computer program instructions 501 are stored. When the computer program instructions 501 are executed by the processor, the steps of any one of the above-mentioned control methods of the exercise device may be implemented.

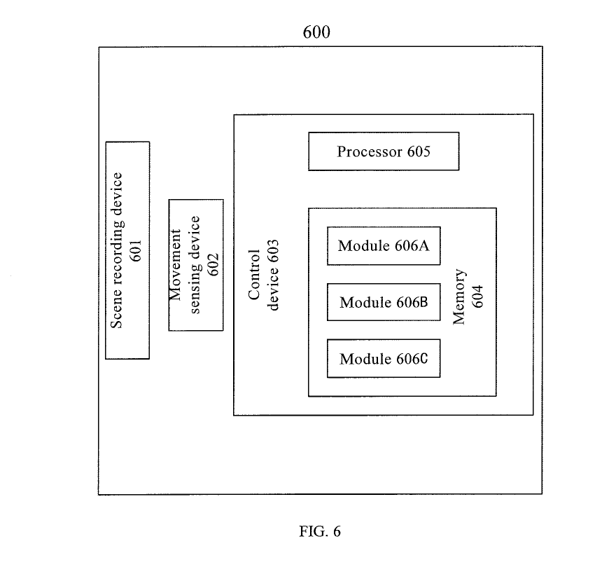

Referring to FIG. 6, a control device 600 of an exercise device is also provided according to an exemplary embodiment of the present disclosure. The control device 600 may include: a scene recording device 601 configured to record exercise parameters of an exerciser in outdoor exercises; a movement sensing device 602 configured to detect body movements of the exerciser; and a control device 603. The control device 603 may include a memory 604, a processor 605 and one or more modules 606A, 606B, 606C stored in the memory 604. The one or more modules 606A, 606B, 606C may be configured to be executed by the processor 605.

For example, the one or more modules 606A, 606B, 606C may include the following instructions: acquiring the exercise parameters of the exerciser in the outdoor exercises according to the mapping relationship between the body movements of the exerciser and the exercise parameters of the exerciser in the outdoor exercises (e.g., the mapping relationship in Table 1); and driving the exercise device to operate according to the acquired exercise parameters.

For example, the scene recording device 601 may be a wearable device such as intelligent glasses or the like, or may be a mobile terminal such as a smart phone or the like. The scene recording device 601 may also be other devices which may record video or capture image, and the embodiments of the present disclosure are not intended to limit the scene recording device. Advantageously, by using the wearable devices such as intelligent glasses, the exerciser may record video or capture image without affecting normal exercises.

For example, the movement sensing device 602 may include a camera or other sensors which may sense the body movements. The movement sensing device 602 may detect the body movements of the exerciser by means of motion capturing techniques.

It should be appreciated that the control device 603 ay be designed with reference to the control device 302 described above with reference to FIG. 3, and is not described in detail herein.

It should be noted that the memory and storage medium in the above embodiments may be at least one readable storage medium having a non-volatile or volatile memory, such as an electrically erasable programmable read only memory (EEPROM), flash memory, and/or hard disk drive.

The processor in the above embodiments may be a single CPU (central processing unit), but may also include two or more processing units. For example, the processor may include a general purpose microprocessor, an instruction processor, and/or associated chipset, and/or a special purpose microprocessor (e.g., an application specific integrated circuit (ASIC). The processor may also include an onboard memory for caching purposes. The computer program instructions may be carried by computer program products connected to the processor. The computer program product may include a computer readable medium having computer program instructions stored thereon.

Although the instructions in the above embodiments are implemented as a computer program and are executed in the processor, however, in alternative embodiments at least one of the instructions may be implemented at least partially as hardware circuitry.

While the disclosed embodiments are described above, the disclosure is merely for the purpose of facilitating understanding of the present disclosure and is not intended to limit the present disclosure. Any modifications and variations may be made to the forms and details of the implementation without departing from the spirit and scope of the disclosure, and the scope of the present disclosure is still intended to be defined by the appended claims.

What is claimed is:

1. A control method of an exercise device, comprising:
detecting body movements of an exerciser,
acquiring exercise parameters of the exerciser in outdoor exercises according to a mapping relationship between the body movements of the exerciser and the exercise parameters of the exerciser in the outdoor exercises; and
driving the exercise device to operate according to the exercise parameters.

2. The control method of claim 1, wherein the exercise parameters comprise at least one selected from exercise scenes, exercise speeds and exercise durations.

3. The control method of claim 2, wherein the mapping relationship comprises a first mapping relationship between the body movements of the exerciser and the exercise scenes of the exerciser in the outdoor exercises, and
wherein the acquiring exercise parameters of the exerciser in outdoor exercises according to a mapping relationship between the body movements of the exerciser and the exercise parameters of the exerciser in the outdoor exercises comprises:
detecting the body movements of the exerciser in sequence, during a pre-recorded video in which the exerciser is exercising in the exercise scenes of the outdoor exercises is played or a pre-captured image in which the exerciser is exercising in the exercise scenes of the outdoor exercises is played; and
acquiring the exercise scenes corresponding to detected body movements of the exerciser according to the first mapping relationship between the body movements of the exerciser and the exercise scenes of the exerciser in the outdoor exercises, in response to detecting the body movements of the exerciser.

4. The control method of claim 3, wherein the driving the exercise device to operate according to the exercise parameters comprises:
driving the exercise device to operate according to the exercise scenes.

5. The control method of claim 3, wherein the exercise parameters comprise multiple sets of exercise parameters, each set of exercise parameters comprising an exercise scene, an exercise speed corresponding to the exercise scene and an exercise duration corresponding to the exercise scenes, and
wherein the driving the exercise device to operate according to the exercise parameters comprises:
according to an acquired exercise scene, determining the exercise speed corresponding to the acquired exercise scene and the exercise duration corresponding to the acquired exercise scene; and
driving the exercise device to operate in a set of exercise parameters, the set of exercise parameters comprising the acquired exercise scene, the exercise speed corresponding to the acquired exercise scene and the exercise duration corresponding to the acquired exercise scene.

6. The control method of claim 2, wherein the mapping relationship comprises a second mapping relationship between the body movements of the exerciser and the exercise speeds of the exerciser in the outdoor exercises, and
wherein the acquiring exercise parameters of the exerciser in outdoor exercises according to a mapping relationship between the body movements of the exerciser and the exercise parameters of the exerciser in the outdoor exercises comprises:
detecting the body movements of the exerciser in sequence, during a pre-recorded video in which the exerciser is exercising in the exercise scenes of the outdoor exercises is played or a pre-captured image in which the exerciser is exercising in the exercise scenes of the outdoor exercises is played; and
acquiring exercise speeds corresponding to detected body movements of the exerciser according to the second mapping relationship between the body movements of the exerciser and the exercise speeds of the exerciser in the outdoor exercises, in response to detecting the body movements of the exerciser.

7. The control method of claim 6, wherein the driving the exercise device to operate according to the exercise parameters comprises:
driving the exercise device to operate according to the exercise speeds.

8. The control method of claim 3, wherein after the acquiring the exercise scenes corresponding to detected body movements of the exerciser according to the first mapping relationship between the body movements of the exerciser and the exercise scenes of the exerciser in the outdoor exercises in response to detecting the body movements of the exerciser, the method further comprises:
calculating a time interval between a moment when a body movement of the exerciser is currently detected and another moment when a body movement of the exerciser was once detected most recently;
determining the time interval as an exercise duration of one of the exercise scenes, the one of the exercise scenes corresponding to the body movement of the exerciser which was once detected most recently; and
recording a third mapping relationship between the exercise scenes and the exercise durations.

9. The control method of claim 8, wherein the driving the exercise device to operate according to the exercise scenes comprises:
driving the exercise device to operate in a first operating state according to an acquired first exercise scene;
determining a first exercise duration corresponding to the first exercise scene according to the third mapping relationship; and
driving the exercise device to operate in a second operating state different from the first operating state, after the exercise device operates in the first operating state for the first exercise duration.

10. An exercise device, comprising:
a movement sensing device configured to detect body movements of an exerciser;
a control device comprising:
a memory;
a processor;
one or more modules stored in the memory and configured to be executed by the processor, the one or more modules comprising instructions:
to acquire exercise parameters of the exerciser in outdoor exercises according to a mapping relationship between the body movements of the exerciser and the exercise parameters of the exerciser in the outdoor exercises; and
to drive the exercise device to operate according to the exercise parameters.

11. The exercise device of claim 10, wherein the exercise parameters comprise at least one selected from exercise scenes, exercise speeds and exercise durations.

12. The exercise device of claim 11, wherein the mapping relationship comprises a first mapping relationship between the body movements of the exerciser and the exercise scenes of the exerciser in the outdoor exercises, and
wherein the instruction to acquire exercise parameters of the exerciser in outdoor exercises according to a mapping relationship between the body movements of the exerciser and the exercise parameters of the exerciser in the outdoor exercises comprises:
detecting the body movements of the exerciser in sequence, during a pre-recorded video in which the exerciser is exercising in the exercise scenes of the outdoor exercises is played or a pre-captured image in which the exerciser is exercising in the exercise scenes of the outdoor exercises is played; and
acquiring the exercise scenes corresponding to detected body movements of the exerciser according to the first mapping relationship between the body movements of the exerciser and the exercise scenes of the exerciser in the outdoor exercises, in response to detecting the body movements of the exerciser.

13. The exercise device according to claim 12, wherein the instruction to drive the exercise device to operate according to the exercise parameters comprises:
driving the exercise device to operate according to the exercise scenes.

14. The exercise device of claim 12, wherein the exercise parameters comprise multiple sets of exercise parameters, each set of exercise parameters comprising an exercise scene, an exercise speed corresponding to the exercise scene and an exercise duration corresponding to the exercise scene, and
wherein the instruction to drive the exercise device to operate according to the exercise parameters comprises:
according to an acquired exercise scene, determining the exercise speed corresponding to the acquired exercise scene and the exercise duration corresponding to the acquired exercise scene; and
driving the exercise device to operate in a set of exercise parameters, the set of exercise parameters comprising the acquired exercise scene, the exercise speed corresponding to the acquired exercise scene and the exercise duration corresponding to the acquired exercise scene.

15. The exercise device of claim 11, wherein the mapping relationship comprises a second mapping relationship between the body movements of the exerciser and the exercise speeds of the exerciser in the outdoor exercises, and
wherein the instruction to acquire exercise parameters of the exerciser in outdoor exercises according to a mapping relationship between the body movements of the exerciser and the exercise parameters of the exerciser in the outdoor exercises comprises:
detecting the body movements of the exerciser in sequence, during a pre-recorded video in which the exerciser is exercising in the exercise scenes of the outdoor exercises is played or a pre-captured image in which the exerciser is exercising in the exercise scenes of the outdoor exercises is played; and
acquiring exercise speeds corresponding to detected body movements of the exerciser according to the second mapping relationship between the body movements of the exerciser and the exercise speeds of the exerciser in the outdoor exercises, in response to detecting the body movements of the exerciser.

16. The exercise device according to claim 15, wherein the instruction to drive the exercise device to operate according to the exercise parameters comprises:
driving the exercise device to operate according to the exercise speeds.

17. The exercise device of claim 12, wherein the one or more modules further comprise instructions:
to calculate a time interval between a moment when a body movement of the exerciser is currently detected and another moment when a body movement of the exerciser was once detected most recently;
to determine the time interval as an exercise duration of one of the exercise scenes, the one of the exercise scenes corresponding to the body movement of the exerciser which was once detected most recently; and
to record a third mapping relationship between the exercise scenes and the exercise durations.

18. The exercise device according to claim 17, wherein the instruction to drive the exercise device to operate according to the exercise scenes comprises:
driving the exercise device to operate in a first operating state according to an acquired first exercise scene;
determining a first exercise duration corresponding to the first exercise scene according to the third mapping relationship; and
driving the exercise device to operate in a second operating state different from the first operating state, after the exercise device operates in the first operating state for the first exercise duration.

19. A control device of an exercise device, comprising:
a scene recording device configured to record exercise parameters of an exerciser in outdoor exercises;
a movement sensing device configured to detect body movements of the exerciser; and
a control device comprising:
a memory;
a processor;
one or more modules stored in the memory and configured to be executed by the processor, the one or more modules comprising instructions:
to acquire exercise parameters of the exerciser in outdoor exercises according to a mapping relationship between the body movements of the exerciser and the exercise parameters of the exerciser in the outdoor exercises; and
to drive the exercise device to operate according to the exercise parameters.

20. A computer readable storage medium on which computer program instructions are stored, wherein the computer program instructions are executed to implement steps of the control method of the exercise device of claim 1.
